(19) United States
(12) Patent Application Publication
(10) Pub. No.: US 2009/0130693 A1
(43) Pub. Date: May 21, 2009

(54) NOVEL BIOMARKERS FOR DIAGNOSIS AND/OR PROGNOSIS OF NEOPLASIAS IN ANIMALS

(76) Inventors: Claudio Bassi, Verona (IT); Pierluigi Mauri, Seregno (IT); Aldo Scarpa, Verona (IT); Claudio Sorio, Verona (IT)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/817,787

(22) PCT Filed: Mar. 6, 2006
(86) PCT No.: PCT/IB06/00478

§ 371 Date: Jul. 16, 2008

(30) Foreign Application Priority Data
Mar. 4, 2005 (IT) VI2005A00059

(51) Int. Cl. G01N 33/574; C12Q 1/02; C07K 14/00

(52) U.S. Cl. 435/7.23; 435/29; 530/350

(57) ABSTRACT

The invention is generally applicable in the field of biomedical engineering, and specifically relates to a method for diagnosis and/or prognosis of neoplasias in animals. The invention further relates to a diagnostic/prognostic kit associated to such method, a reagent for the preparation of such kit and the use of particular biomarkers in such method and/or kit. The method comprises at leas the steps of: drawing at least one sample from the patient and determining the amount of a biomarker in said at least one sample drawn from the patient. The biomarker is a protein released by pancreatic cells.

### FIELD OF THE INVENTION

This invention is generally applicable in the field of biomedical engineering, and specifically relates to a method for diagnosis and/or prognosis of neoplasias in animals.

The invention further relates to a kit for diagnosis and/or prognosis of neoplasias in animals, a reagent for the preparation of such kit and the use of particular biomarkers in such method and/or kit.

### BACKGROUND ART

Tumor cells modify and interact with their microenvironment by secreting a variety of proteins, including growth factors, extracellular matrix-degrading proteinases involved in tumor invasion, and cell motility factors that support cell migration and metastasis.

Furthermore, a number of additional factors were found to be involved in immunological escape, tumor invasion and angiogenesis, such as immunoregulatory cytokineses and molecules regulating both cell-to-cell and cell-to-substrate interactions (§ 1-3).

Pancreatic adenocarcinoma is a lethal disease, with an expected survival rate of less than 5% at 24 months after diagnosis. The identifications of proteins released by tumor cells may be useful both to understand the interaction between the tumor and the host organ and to find new methods for diagnosis, prognosis or treatment.

In recent years molecular analysis of cancer has been performed using DNA-microarrays, providing global profiles of transcription that reflect the origins (§ 4-6), stage of development (§ 7) and drug sensitivity (§ 7) of tumor cells. This approach has been also used to identify putative secreted proteins by the analysis of mRNAs bound to membrane-associated polysomes (§ 8). Nevertheless, mRNA-based analysis is an indirect approach to molecular analysis of cancer and necessitates validation at the protein level.

One step forward has been made through direct approaches based on proteome analysis (§ 9-11), although several technical problems arise when analyzing of complex protein mixtures (§ 11). The classical proteomic approach is based on two-dimensional gel electrophopresis, known as “2DG”, in which the proteins of interest are isolated and identified by mass spectrometry.

Thanks to the above mentioned method, and to the other approaches described above, various studies have been conducted (§ 12-§ 24) to identify pancreatic tumor “detecting” proteins. These include MMP-1, MMP-7, TIMP1, SERPINE2, TGFBI, MAC-2BP, clusterine, glycerol-3-phosphate dehydrogenase, syndecan-1, TSP-1 and uPA.

Many of these proteins were found to be involved in cell-cell and cell-extracellular matrix adhesion, as well as in extracellular matrix degradation and remodeling, and thus promote invasion and metastasis.

Nevertheless, this solution still has a few apparent drawbacks. While the 2DG approach has a relatively high resolution, this is limited by the difficulty to detect certain classes of proteins.

These include the cell membrane proteins, due to their low solubility in the gel-electrophoresis buffer, proteins of excessively low or high molecular weight, i.e. of less than 10 kDa or more than 200 kDa respectively, and proteins with an extreme isoelectric point, i.e. less than 4 or more than 9. Furthermore, the 2DG analysis cannot detect proteins in small amounts (§ 25).

### SUMMARY OF THE INVENTION

The Applicant has been engaged in cancer-related research and has surprisingly found novel biomarkers for use in the diagnosis and/or prognosis of neoplasias in animals.

Therefore, the present invention is aimed at providing a method for diagnosis and/or prognosis of neoplasias in animals, as defined in claim 1, which comprises at least the steps of drawing at least one sample from the patient and determining the amount of a biomarker in said at least one sample drawn from the patient, wherein said biomarker is a protein released from pancreatic cells. Advantageously, the biomarker may be selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69, whereas the neoplasia may be a tumor of the pancreas.

Thanks to these proteins the reliability of diagnosis and/or prognosis of neoplasias, particularly the carcinoma of the pancreas will be greatly enhanced.

Suitably, the neoplasia may be selected from the group consisting of tumors of the breast, esophagus, head and neck, liver, lung, gastrointestinal tract, prostate, skin, kidney and/of urogenital system, metastases, micrometastases or a combination thereof.

Preferably, the animal mentioned above is a mammal, and more preferably a human.

Suitably, the sample may be a body fluid, preferably selected from the group consisting of blood, plasma, serum, urine, sperm, interstitial fluid, spinal fluid or a combination thereof.

Advantageously, the concentration of the above mentioned biomarker may be compared with known concentrations of the same biomarker, detected on samples of the same nature from different animals not suffering from neoplasia, preferably animals having a benign tumor.

Conveniently, the prognosis and/or diagnosis of the neoplasia may be determined by comparing the concentration of said biomarker detected on samples drawn from the same patient.

According to another aspect of the invention, a kit is provided for diagnosis and/or prognosis of neoplasias in animals, as defined in claim 11, which comprises a detectable agent linked to a biomarker, wherein said biomarker is a protein released by pancreatic cells.

Advantageously, the biomarker will be selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

Conveniently, the detectable agent may be selected from the group consisting of an anti-biomarker antibody, preferably of the monoclonal or polyclonal type, a receptor for said biomarker, or functional fragments, or a combination thereof.

Advantageously, the agent may be detectable by measuring chromatography, electrical capacitance, fluorescence, luminescence, mass, molecular weight, radioactivity or a combination thereof.

According to another aspect of the invention, a reagent is provided for diagnosis and/or prognosis of neoplasias, as defined in claim 16, which comprises a detectable agent linked to a biomarker, wherein said biomarker is a protein released by pancreatic cells.

Conveniently, the biomarker may be selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

In accordance with a further aspect of the invention, there is provided the use of proteins released by pancreatic cells as biomarkers for diagnosis and/or prognosis of neoplasias, as defined in claim 18.

Conveniently, the biomarker may be selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

### BRIEF DESCRIPTION OF THE FIGURES

Further features and advantages of the invention will be more readily apparent from the detailed description of a few preferred non exclusive embodiments of a method according to the invention, which are shown as a non limiting example with the help of the annexed figures, in which:

FIG. 1 shows CSPG/Versican and Mac25/Angiomodulin expression in pancreatic adenocarcinoma.

CSPG2Nersican, frame A: Immunohistochemistry of primary pancreatic adenocarcinoma shows strongly positive peritumoral stroma, while tumor cells are immunonegative (arrow).
Frame B: the same pattern of reactivity detected in Suit-2 cells implanted in nu/nu mice in a Matrigel® matrix. This shows strong immunostrain with anti-versican antibody at the cell-matrix interface, while tumor cells expression is undetectable. This demonstrates that cancer cells produce and immediately release the protein.
Frame C: RT-PCR analysis shows the presence of versican transcripts in five of the six cell lines, T3M4 being negative. Actin expression is shown to demonstrate equal amounts of starting RNA.
Mac25/Angiomodulin, frame D: tumor cells in primary pancreatic adenocarcinoma show strong cytoplasmic immunostain.
Frame E: Immunohistochemistry of Suit-2 cells implanted in nu/nu mice in Matrigel® matrix shows immunostain with anti-mac25 antibody in both the cytoplasm of cells and the matrix.
Frame F: Western blot analysis of supernatants shows that angiomodulin is released from five of the six tumor cell lines; anti-cdc42 is the negative control antibody, and Ponceau staining indicates the relative amount of proteins loaded on each line.

FIG. 2 shows an example of MAProMA virtual 2D map obtained from the data of Tables 1 and 2. A color is assigned to each spot (corresponding to an assigned protein), according to the score value obtained by SEQUEST software:

yellow <20, blue from 20 to 60 and red >60. The top right inset shows a plot limited to proteins having a molecular weight of less than 100 kDa.

### DETAILED DESCRIPTION OF A FEW PREFERRED EMBODIMENTS

As shown in the sections below, the Applicant surprisingly identified 46 proteins (see Table 1 and 2) which may be related to relevant tumor cell features, such as angiogenesis and modification of the extracellular environment.

Twenty-one of these were classified as secreted proteins according to Human Protein reference and GeneCards™ databases, while the remaining ones were classified as cytoplasmic proteins.

Eleven of the twenty-one proteins released in the medium (MMP-1, MMP-7, TIMP1, SERPINE2, TGFBI, MAC-2BP, clusterine, glycerol-3-phosphate dehydrogenase, syndecan-1, TSP-1 and uPA) have already been associated with pancreatic tumor, indicating the validity of the selected approach.

The remaining ten proteins have never been associated to pancreatic tumor heretofore. These include: CSPG2/versican, Mac25/angiomodulin, IGFBP-1, HSPG2/perlecan, syndecan 4, FAM3C, APLP2, cyclofilin B, beta2 microglobulin and ICA69.

The demonstration that CSPG2/versican is produced and immediately released by pancreatic tumor cells challenges a technical-scientific prejudice. A number of studies allege that fibroblasts of stroma cancer are the sole or predominant source of these molecules (§ 35-38).

Mac25/angiomodulin is a member of the insulin-like growth factor binding protein (IGFBP), and has never been associated with pancreatic tumor heretofore.

The finding that Mac25/angiomodulin is produced in vivo by endothelial or tumor cells, even though it is associated to a number of other phenomena (§ 39-41) fulfils a long-felt need in the technical-scientific community.

IGFBP-1 is another member of the IGFBP family and has been recently associated with increased risk of hematological malignancy (§ 42) but never with pancreatic carcinoma heretofore.

HSPG2/perlecan is a major heparan sulfate proteoglycan component of basement membranes and connective tissues. Suppression of its expression is known to inhibit tumor growth and neovascularization in human colon carcinoma xenografts and mouse melanoma allografts (§ 43) but has never been associated with pancreatic tumor heretofore.

Cells transfected with syndecan-4 are further known to be able to bind collagen and show reduced invasive capability (§44) but such protein has been never associated to pancreatic carcinoma heretofore.

FAM3C is a member of a recently cloned cytokine-like gene family (§ 45), and has never been associated to pancreatic tumor heretofore.

The remaining proteins (APLP2, cyclophilin B, beta2 microglobulin and ICA69) have been reported to be expressed by pancreatic tumor cell lines (§ 46-49), but no one has never shown their direct relationship with pancreatic cancer heretofore.

Those of normal skill in the art may wholly reproduce the invention as described in sections below by way of non limiting example of the invention.

As is known, the invention is susceptible of a many changes and variants within the inventive principle disclosed in the annexed claims. All the steps, features, compounds and compositions may be replaced by equivalents, and several different materials may be used as needed, without departure from the scope of the invention.

While the invention has been described with particular reference to the annexed figures, such description shall be intended to only improve the intelligibility of the invention and not to limit the claimed scope in any manner.

### Materials and Methods

A protein identification method known as “MudPIT”, multidimensional protein identification technology (§ 13) was used, which incorporates two-dimensional capillary chromatography as well as an automated tandem mass spectrometry (2DC-MS/MS).

This technique uses the peptides generated from enzymatic digestion of a complex protein mixture, by first separating them by means of two micro-HPLC columns, and then directly analyzing the eluted peaks by tandem mass spectrometry. The identification of the corresponding proteins is then obtained through an automated database search with appropriate software, such as the SEQUEST algorithm for mass spectra data handling (§ 13-15).

Harvest of supernatants Suit-2, IMIM-PC1, IMIM-PC2, T3M4, BI and MCC1 pancreatic tumor cell lines have been used. The first five cell lines have been described in Moore et al (§ 26), and the last one has been established at the Pathology laboratory of the University of Verona (§ 27). The supernatants were harvested following the protocol of Krachmarova et al (§ 28). Cells were grown to 80% confluency in 150 cm2 flasks with complete medium (RPMI 1640, 2 mM glutamine, 10% FBS), gently washed 6 times with serum-free medium (30 mM) and left in 12 ml of serum-free medium for additional 18 h. The supernatants were ultracentrifuged at 100,000 g for 2 h at 4° C. and subjected to analysis. For cell activation analysis, Suit-2 cells were treated with 100 ng/ml phorbol myristate acetate (PMA) and 100 nM ionomicin.

Enzymatic digestion of protein samples Sequencing grade modified trypsin was added to 50 μl of conditioned medium containing 1 μg protein at a 1:50 enzyme:protein ratio (wt:wt) in 100 mM ammonium bicarbonate, pH 8.0 and incubated at 37° C. overnight. The reaction was stopped by acidification with trifluoroacetic acid. A second aliquot of each sample was digested with pepsin at 1:50 enzyme:protein ratio (wt:wt) in 100 mM ammonium acetate pH 3.0 at room temperature for 4 h and immediately analyzed. 10 μl of the peptide mixture so obtained were injected directly into the 2DC-MS/MS.

Two-dimensional capillary chromatography—tandem mass spectrometry (2DC-MS/MS) analysis 10 μl of the peptide mixture obtained from the digestion of the protein samples, were analyzed by means of two-dimensional microchromatography coupled with an ion trap mass spectrometer, using the ProteomeX system equipped with Bioworks 3.1 as graphical interface for data handling. Particularly, peptide mixtures were first separated by means of ion-exchange chromatography (Biobasic-SCX column, 5 μm, 0.3 i.d.×150 mm) through seven steps of increasing ammonium chloride concentration (0, 50, 100, 150, 200, 300, and 600 mM). Each predetermined salt concentration step was directly loaded onto the reversed phase column (Biobasic-C₁₈, 0.180 i.d.×100 mm, ThermoHypersil, Bellofonte, Pa.) and separated with an acetonitrile gradient. The following were used: eluent A, 0.1% formic acid in water; eluent B, 0.1% formic acid in acetonitrile; the gradient profile was 5% B for 3 min followed by 5 to 50% B within 40 min.

Peptides eluted from the C₁₈ column were analyzed directly with an ion trap LCQ_{XP} mass spectrometer equipped with a metal needle (10 μm i.d.). The heated capillary was held at 160° C., ion spray 3.2 kV and capillary voltage 67 V. The spectrum was acquired in positive mode (in the range of 400-1600 m/z) using dynamic exclusion for MS/MS analysis (collision energy 35%).

Data handling of MS results Using the SEQUEST algorithm, the experimental mass spectrum produced was related to the peptide sequence obtained by comparison with the theoretical mass spectrum in the human protein database downloaded from the NCBI website. For peptide matching, the following limits were used: Xcorr scores of more than 1.5 for simple charge peptide ions, of more than 2.0 and 2.5 for double and triple charge ions respectively. The output data obtained from SEQUEST software were treated with the MAProMA (Multidimensional Algorithm Protein Map) algorithm for comparison of protein lists, evaluation of relative abundances, and plotting of virtual 2D maps (§ 29).

Western blotting. Proteins were precipitated from 10 ml of conditioned, serum-free medium by drop-wise addition of 10% (final concentration) tricloroacetic acid with stirring at 4° C. The sample was then centrifuged at 3000 g for 60 min and washed 3 times with an excess of an acetone: methanol (8:1) mixture.) The pellet was air-dried, resuspended in SDS buffer, subjected to SDS-PAGE and Western blotting. Anti-angiomodulin antibody 88 (§ 30) and an anti-versican antibody (clone 2-B-1) were used at 1 μg/ml. The secondary antibody was a rabbit anti-mouse-HRP, and the signal was detected using a chemiluminescence detection kit.

Immunohistochemistry 5 μm paraffin sections were stained using anti-angiomodulin at 5 μg/ml, and anti-versican at 0.5 μg/ml, as described in Cattaneo et al (§ 31).

Xenografting Suit-2 cells in nude mice 2*10⁶ Suit-2 cells were resuspended in 0.4 ml of Matrigel® and inoculated subcutaneously in the flank of four weeks old nu/nu Swiss mice weighing 18-22 g. After 1 wk, the implant was removed, fixed in 10% buffered formalin, paraffin-embedded, and sectioned for immunohistochemistry.

Reverse Transcription-Poltmerase Chain Reaction (RT-PCR). RNA was prepared using the Trizol® extraction kit. One μg of total RNA was reverse transcribed in 20 μl with 100 ng of random hexamers and 200 U of SuperScript II® at 42° C. for 1 h. Polymerase chain reaction was performed as described in Cattaneo et al (§ 31). Amplification of β-actin mRNA was performed for 25 cycles on cDNA as control. The primers to amplify versican were: 5′-GGC TTT GAC CAG TGC GAT TAC-3′ and 5′-CCA GCC ATA GTC ACA TGT CTC-3′.

Gelatin/zymography for metalloprotease activity Supernatants were centrifuged for 10 min at 14,000 g to remove cell debris. Five μl of 5×SDS sample buffer (5% SDS, 0.5 M Tris-HCl pH 6.8, 25% glycerol) were added to 20 μl of supernatant. The sample was run on an SDS-PAGE gel containing 1 mg/ml gelatin. The gel was washed twice (20 min/cycle) with 2.5% Triton X-100 at room temperature, incubated in 200 ml of activation buffer (10 mM Tris-HCl, 1.25% Triton X-100, 5 mM CaCl₂, 1 μM ZnCl₂) overnight at 37° C., stained with Coomassie blue and destained with methanol: acetic acid:water (50:10:40).

### Results

MudPIT analysis of serum-free supernatants of resting and phorbol-ester activated Suit-2 cell lines identified 46 proteins (Tables 1 and 2). The results were validated for certain proteins by analyzing a panel of tumor cell lines. Evidence that the latter release these proteins in vivo was obtained by immunohistochemistry on both primary pancreatic tumors and in a model consisting of Suit-2 cells embedded in an amorphous matrix and implanted in athymic mice. MudPIT analysis further proved to reveal changes in the amount of secreted proteins after phorbol-ester activation of cells, as reflected by the SEQUEST software score values.

Determination of optimal conditions and time point for collection of supernatants To determine the optimal treatment and supernatant collection times, the 3DC-MS/MS proteomic approach was applied to several different solutions. Such optimal conditions (e.g. determination of the minimum number of cytosolic proteins susceptible of cell damage) turned out to be the protocol of Kratchmarova et al. with minor modifications, consisting in a gentle wash followed by 18 h incubation in protein-free medium

Identification of proteins released by Suit-2 cells Each 2DC-MS/MS analysis of digested samples produced seven reversed-phase chromatograms, which corresponded to the seven SCX column salt steps of increasing ammonium concentration (0, 50, 100, 150, 200, 300, and 600 mM). The peptides eluted from the columns were immediately directed into the mass spectrometer where they were ionized, mass selected, and fragmented. This procedure allowed to identify several peptides that were then associated with their respective proteins by the SEQUEST software.

MudPIT identified 30 proteins released by resting pancreatic cancer cells The MudPIT analysis of the supernatant from Suit-2 cells reproducibly identified, from 4 independent cell cultures, the 30 proteins listed in Table 1, where their putative cellular location according to public databases is also reported. Some of these proteins have never been associated with pancreatic tumor heretofore, including CSPG2/versican and Mac25/angiomodulin. MudPIT data was validated for two of them, CSPG2/versican e Mac25/angiomodulina, because these were present in larger amounts in samples, with respect to the number of peptides detected in supernatants.

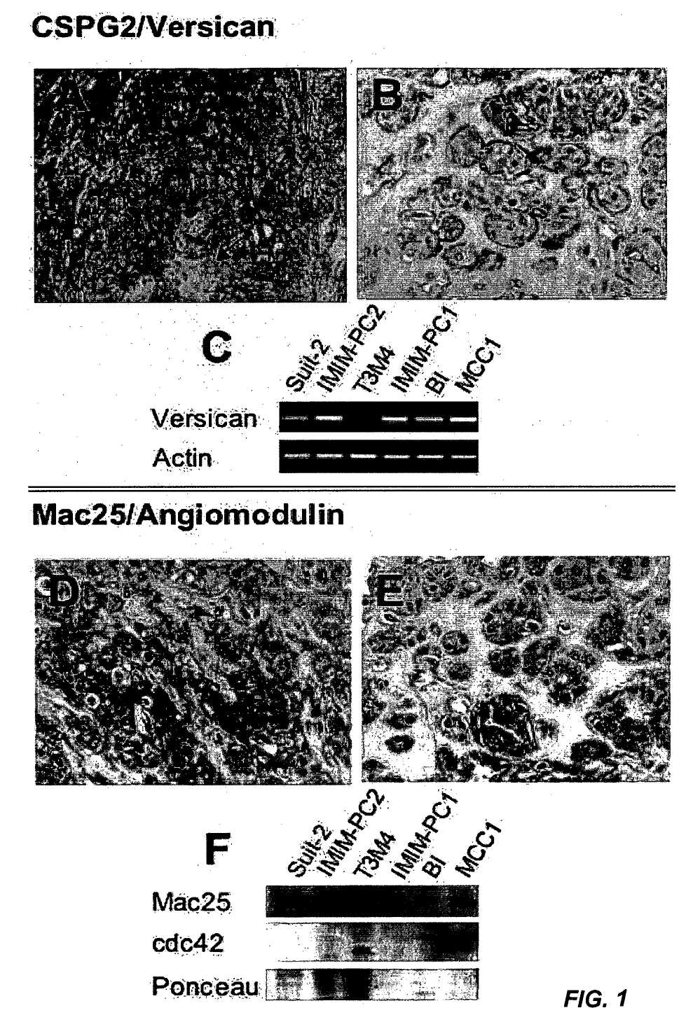

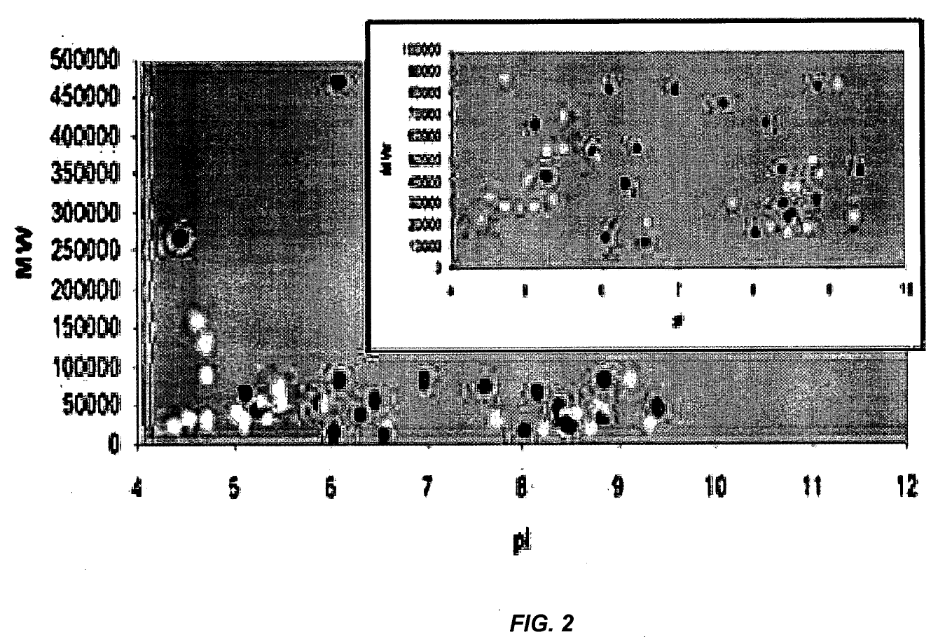

CSPG2/versican is released by Suit-2 and primary pancreatic adenocarcinoma cells CSPG2/versican mRNA was detected by RT-PCR in five of the six cell lines under test (FIG. 1C). Among primary pancreatic tumors a large amount of CSPG2/versican was detected in the desmoplastic stroma, while cancer cells were immunonegative (FIG. 1A). To demonstrate that CSPG2/versican is released by cancer cells in vivo, an experimental model was set up, consisting in Suit-2 cells resuspended in an amorphous matrix (Matrigel®), xenografted in the flank of nu/nu mice and allowed to proliferate for one week. The implant was then immunostained with a versican antibody. The analysis clearly showed that secretion does occur, as the prmteoglycan accumulates at the interface between cells and the matrix, before diffusing in the matrix itself (FIG. 1B). The cells themselves did not stain with the antibody, indicating a low intracellular accumulation of the proteoglycan compatible with rapid release.

Mac25/angiomodulin is a major secreted protein in pancreatic tumor and is overexpressed in primary pancreatic tumors. Western blot analysis showed that five of the six pancreatic tumor cell lines released Mac25 in the supernatant (FIG. 1F), and the presence of the protein within the cells was further confirmed by immunoprecipitation and Western blotting of cell lysates (not shown). Immunohistochemistry showed that Mac25 was clearly expressed in both primary tumor cells (FIG. 1D) and xenografted Suit-2 cells (FIG. 1E). In normal pancreas, Mac25 was expressed in the insulae of Langerhans, while a faint signal was present in small ducts (not shown).

Additional proteins were identified upon phorbol ester/ionophor activation of tumor cells After phorbol ester-ionomycin activation, the MudPIT analysis of the supernatants from Suit-2 cells reproducibly identified, from 4 independent cell cultures, the 16 proteins listed in Table 2, in addition to those detected in resting conditions (Table 1). Among these, six proteins were classified as secreted in public databases. Moreover, it was noticed that several proteins identified in supernatants of both resting and activated conditions had varied score values assigned thereto by SEQUEST software. This suggests that these score values might be related to changes in the amount of proteins detected in the two conditions.

Virtual 2D map of identified proteins To visualize the SEQUEST output data in a user-friendly format, the MAProMA (multidimensional algorithm protein map) software was developed, which automatically plots molecular weight (MW) vs. isoelectric point (pl) for each identified protein, as shown in FIG. 2.

The representation of MudPIT results on a 2D map immediately highlights proteins having a very high molecular weight and/or pl, as is the case for the two proteins in FIG. 2 having a MW of about 260 and 460 kDa, which correspond to CSPG2/versican and HSPG2/perlecan, respectively.

TABLE 1
Proteins identified in the serum-free supernatant of resting Suit-2 cells

| NCBI | | | % | | MW | Cellular |
|---|---|---|---|---|---|---|
| GI number | Protein name | Peptides | Sequence | pI | (Da) | Location |
| 4758082 | CSPG2/versican (Chondroitin sulfate proteoglycan 2) | 9 | 7 | 4.45 | 265050 | S |
| 4505215 | MMP1 (Matrix metalloproteinase 1 preproprotein) | 6 | 16 | 6.47 | 54008 | S |
| 5031863 | Mac-2 BP (Mac-2-binding protein; serum protein 90K) | 5 | 13 | 5.13 | 65332 | S |
| 7427517 | HSPG2/perlecan (Heparan sulfate proteoglycan 2) | 5 | 2 | 6.10 | 469388 | S |
| 2135211 | Mac25/angiomodulin/IGFBP-rP1/IGFBP7 | 4 | 19 | 8.40 | 28756 | S |
| 87509 | SERPINE2 (Protease Nexin 1) | 4 | 14 | 9.42 | 43961 | S |
| 4507467 | TGFBI (Transforming growth factor, beta-induced) | 4 | 10 | 7.62 | 74681 | S |
| 4502905 | Clusterin (Apolipoprotein J) | 3 | 11 | 5.89 | 52495 | S |
| 4505219 | MMP7 (Matrix metalloproteinase 7) | 3 | 16 | 7.73 | 29678 | S |
| 7661714 | FAM3C (Predicted osteoblast protein) | 3 | 23 | 8.52 | 24681 | S |
| 4504615 | IGFBP-1 (Insulin-like growth factor binding protein 1) | 1 | 6 | 5.11 | 27905 | S |
| 4507508 | TIMP1 (Tissue inhibitor of metalloproteinase 1) | 1 | 5 | 8.46 | 23171 | S |
| 4502147 | APLP2 (Amyloid beta (A4) precursor-like protein 2) | 2 | 5 | 4.73 | 86956 | M/S |
| 7446012 | Glycerol-3-phosphate dehydrogenase | 4 | 9 | 6.98 | 80815 | C/S |
| 16162032 | Cyclophilin B (Hypothetical protein XP_057192) | 3 | 19 | 9.33 | 22743 | C/S |
| 1346343 | CK 1 (Keratin, type II) | 4 | 9 | 8.16 | 66018 | C |
| 4501885 | Beta actin | 3 | 18 | 5.29 | 41737 | C |
| 7669492 | (Glyceraldehyde-3-phosphate dehydrogenase | 2 | 11 | 8.57 | 36054 | C |
| 4501883 | Alpha 2 actin | 2 | 9 | 5.24 | 42010 | C |
| 5031857 | Lactate dehydrogenase A | 1 | 5 | 8.44 | 36689 | C |
| 4826908 | Phosphoinositide-3-kinase | 1 | 3 | 6.10 | 81624 | C |
| 17380170 | Poly(A) polymerase gamma | 1 | 3 | 9.12 | 82806 | C |
| 4826898 | Profilin 1 | 1 | 11 | 8.44 | 15055 | C |
| 4507553 | Tropomodulin | 1 | 6 | 5.03 | 40570 | C |
| 9628503 | Regulatory protein E2 | 1 | 4 | 8.39 | 44333 | C |
| 10181098 | Choline acetyltransferase isoform | 1 | 2 | 8.86 | 82609 | C |
| 7662130 | KIAAO443 gene product | 1 | 2 | 4.64 | 156837 | C |
| 5032057 | S100 calcium-binding protein A11 | 2 | 24 | 6.56 | 11741 | C/N |
| 5032221 | Voltage-dependent anion channel 3 | 2 | 8 | 8.84 | 30659 | C (mit) |
| 1172554 | Voltage-Dependent Anion-Selective Channel Protein 2 | 5 | 23 | 6.32 | 38093 | C (mit) |

Key: Peptides, number of peptides identified; % Sequence, percent of protein coverage; pI, Isoelectric point; MW, molecular weight.
Cellular location is based on the Human Protein Reference (http://www.hprd.org) and GeneCards ™ database (http://bioinformatics.weizmann.ac.il). Key: M, membrane; S, secreted; C, cytoplasmic; mit, mitochondrium, N, nucleus.

TABLE 2
Proteins identified in the supernatant of Suit-2 cells after activation
by PMA-ionomycin treatment, in addition to those listed in Table 1.

| NCBI | | | % | | | Cellular |
|---|---|---|---|---|---|---|
| GI number | Protein name | Peptides | Sequence | pI | MW | Location |
| 137112 | uPA (Urokinase-type plasminogen activator precursor) | 4 | 13 | 8.78 | 48526 | S |
| 135717 | TSP1 (Thrombospondin 1) | 4 | 6 | 4.71 | 129413 | S |
| 4757826 | Beta-2-microglobulin | 2 | 16 | 6.05 | 13715 | M/S |
| 4506861 | Syndecan 4 (amphiglycan, ryndocan) | 1 | 7 | 4.39 | 21608 | M/S |
| 135155 | Syndecan-1 | 1 | 6 | 4.53 | 32477 | M/S |
| 729800 | ICA69 (Islet cell autoantigen p69) | 1 | 4 | 5.50 | 54673 | C/S |
| 105583 | CD44E protein, epithelial | 1 | 3 | 5.29 | 53597 | M |
| 5031635 | Cofilin 1 | 2 | 27 | 8.22 | 18503 | C |
| 285975 | Human rab GDI | 2 | 8 | 5.94 | 50664 | C |
| 462325 | Heat shock 70 kDa protein 1 (HS71) | 2 | 6 | 5.48 | 70053 | C |
| 4507953 | Tyrosine 3-monooxygenase/tryptophan 5- | 1 | 12 | 4.73 | 27745 | C |
| | monooxygenase activation protein (14-3-3zeta) | | | | | |
| 7657667 | Ubiquitin-like 4 | 1 | 10 | 8.71 | 17777 | C |
| 107560 | Ras inhibitor (clone JC265) | 1 | 4 | 5.49 | 54385 | C |
| 4503439 | E2F transcription factor 6 | 1 | 6 | 5.35 | 31844 | C/N |
| 11359932 | Hypothetical protein DKFZp434C0917 | 1 | 16 | 8.03 | 15551 | ? |
| 743789 | Probable succinate-CoA ligase (GDP-forming) | 1 | 10 | 6.60 | 20895 | ? |

Key: Peptides, number of peptides identified; % Sequence, percent of protein coverage; pI, Isoelectric point; MW, molecular weight.
Cellular location is based on the Human Protein Reference (http://www.hprd.org) and GeneCards ® database (http://bioinformatics.weizmann.ac.il). Key: M, membrane; S, secreted; C, cytoplasmic; mit, mitochondrium, N, nucleus.

### REFERENCES

The following documents, set forth hereunder as references, are designated hereinbefore by symbol §, and a number corresponding to the following number list, both enclosed in brackets.
1. Barnard, J. A. (2003), Peptide Growth Factors. In Gastrointestinal Cancers, (Rustgi, A. K., ed) pp. 33-54, Saunders, New York
2. Liotta, L. A., and Paweletz, C. P. (2002), Invasion and Metastasis. In The Cancer Handbook, (Alison, M., ed) Vol. 1 pp. 225-234, Nature Publishing Group, London
3. Castells, A., and Rustgi, A. K. (2003), Tumor Invasion and Metastasis. In Gastrointestinal Cancers, (Rustgi, A. K., ed) pp. 69-79, Saunders, New York
4. Golub, T. R., Slonim, D. K., Tamayo, P., Huard, C., Gaasenbeek, M., Mesirov, J. P., Coller, H., Loh, M. L., Downing, J. R., Caligiuri, M. A., et al (1999), Molecular classification of cancer: class discovery and class prediction by gene expression monitoring, Science 286, 531-537
5. Ross, D. T., Scherf, U., Eisen, M. B., Perou, C. M., Rees, C., Spellman, P., Iyer, V., Jeffrey, S. S., Van de Rijn, M., Waltham, M., et al (2000), Systematic variation in gene expression patterns in human cancer cell lines, Nat Genet 24, 227-235
6. Bittner, M., Meltzer, P., Chen, Y., Jiang, Y., Seftor, E., Hendrix, M., Radmacher, M., Simon, R., Yakhini, Z., Ben-Dor, A., et al (2000), Molecular classification of cutaneous malignant melanoma by gene expression profiling, Nature 406, 536-540
7. Perou, C. M., Sorlie, T., Eisen, M. B., van de Rijn, M., Jeffrey, S. S., Rees, C., A., Pollack, J. R., Ross, D. T., Johnsen, H., Akslen, L. A., et al (2000), Molecular portraits of human breast tumours, Nature 406, 747-752
8. Diehn, M., Eisen, M. B., Botstein, D., and Brown, P. O. (2000), Large-scale identification of secreted and membrane-associated gene products using DNA microarrays, Nat Genet 25, 58-62
9. Cravatt, B. F., and Sorensen, E. J. (2000), Chemical strategies for the global analysis of protein function, Curr Opin Chem Biol 4, 663-668 Griffin, T. J., and Aebersold, R. (2001), Advances in proteome analysis by mass spectrometry, J Biol Chem 276, 45497-45500
10. Corthals, G. L., Wasinger, V. C., Hochstrasser, D. F., and Sanchez, J. C. (2000), The dynamic range of protein expression: a challenge for proteomic research, Electrophoresis 21, 1104-1115
11. Ellenrieder, V., Alber, B., Lacher, U., Hendler, S. F., Menke, A., Boeck, W., Wagner, M., Wilda, M., Friess, H., Buchler, M., et al (2000), Role of MT-MMPs and MMP-2 in pancreatic cancer progression, Int J Cancer 85, 14-20
12. Kitamura, N., Iwamura, T., Taniguchi, S., Yamanari, H., Kawano, M. A., Hollingsworth, K., and Setoguchi, T. (2000), High collagenolytic activity in spontaneously highly metastatic variants derived from a human pancreatic cancer cell line (SUIT-2) in nude mice, Clin Exp Metastasis 18, 561-571
13. Bramhall, S. R., Neoptolemos, J. P., Stamp, G. W., and Lemoine, N. R. (1997), Imbalance of expression of matrix metalloproteinases (MMPs) and tissue inhibitors of the matrix metalloproteinases (TIMPs) in human pancreatic carcinoma, J Pathol 182, 347-355
14. Buchholz, M., Biebl, A., Neebetae, A., Wagner, M., Iwamura, T., Leder, G., Adler, G., and Gress, T. M. (2003), SERPINE2 (protease nexin 1) promotes extracellular matrix production and local invasion of pancreatic tumors in vivo, Cancer Res 63, 4945-4951
15. Schneider, D., Kleeff, J., Berberat, P. O., Zhu, Z., Korc, M., Friess, H., and Buchler, M. W. (2002), Induction and expression of betaig-h3 in pancreatic cancer cells, Biochim Biophys Acta 1588, 1-6
16. Tinari, N., D'Egidio, M., lacobelli, S., Bowen, M., Starling, G., Seachord, C., Darveau, R., and Aruffo, A. (1997), Identification of the tumor antigen 90K domains recognized by monoclonal antibodies SP2 and L3 and preparation and characterization of novel anti-90K monoclonal antibodies, Biochem Biophys Res Commun 232, 367-372
17. Kunzli, B. M., Berberat, P. O., Zhu, Z. W., Martignoni, M., Kleeff, J., Tempia-Caliera, A. A., Fukuda, M., Zimmermann, A., Friess, H., and Buchler, M. W. (2002), Influences of the lysosomal associated membrane proteins (Lamp-1, Lamp-2) and Mac-2 binding protein (Mac-2-BP) on the prognosis of pancreatic carcinoma, Cancer 94, 228-239
18. Xie, M. J., Motoo, Y., Su, S. B., Mouri, H., Ohtsubo, K., Matsubara, F., and Sawabu, N. (2002), Expression of clusterin in human pancreatic cancer, Pancreas 25, 234-238
19. Huang, C. J., Severin, E., and Blum, M. (1994), Flow-cytometric determination of dehydrogenase activities in primary human gastrointestinal tumor cell lines, Anal Cell Pathol 6, 93-103
20. Conejo, J. R., Kleeff, J., Koliopanos, A., Matsuda, K., Zhu, Z. W., Goecke, H., Bicheng, N., Zimmermann, A., Korc, M., Friess, H., et al (2000), Syndecan-1 expression is up-regulated in pancreatic but not in other gastrointestinal cancers, lnt J Cancer 88, 12-20
21. Kasper, H. U., Ebert, M., Malfertheiner, P., Roessner, A., Kirkpatrick, C. J., and Wolf, H. K. (2001), Expression of thrombospondin-1 in pancreatic carcinoma: correlation with microvessel density, Virchows Arch 438, 116-120
22. Albo, D., Berger, D. H., and Tuszynski, G. P. (1998), The effect of thrombospondin-1 and TGF-beta 1 on pancreatic cancer cell invasion, J Surg Res 76, 86-90
23. Cantero, D., Friess, H., Deflorin, J., Zimmermann, A., Brundler, M. A., Riesle, E., Korc, M., and Buchler, M. W. (1997), Enhanced expression of urokinase plasminogen activator and its receptor in pancreatic carcinoma, Br J Cancer 75, 388-395
24. Gygi, S. P., Corthals, G. L., Zhang, Y., Rochon, Y., and Aebersold, R. (2000), Evaluation of two-dimensional gel electrophoresis-based proteome analysis technology, Proc Natl Acad Sci USA 97, 9390-9395
25. Moore, P. S., Sipos, B., Orlandini, S., Sorio, C., Real, F. X., Lemoine, N. R., Gress, T., Bassi, C., Kloppel, G., Kalthoff, H., et al (2001), Genetic profile of 22 pancreatic carcinoma cell lines. Analysis of K-ras, p53, p16 and DPC4/Smad4, Virchows Arch 439, 798-802
26. Sorio, C., Capelli, P., Lissandrini, D., Moore, P., Balzarini, P., Falconi, M., Zamboni, G., and Scarpa, A. (2004), A unique cell line and xenograft model of in situ mucinous cystic carcinoma of the pancreas, Virchows Arch, in press
27. Kratchmarova, I., Kalume, D. E., Blagoev, B., Scherer, P. E., Podtelejnikov, A. V., Molina, H., Bickel, P. E., Andersen, J. S., Fernandez, M. M., Bunkenborg, J., et al (2002) A proteomic approach for identification of secreted proteins during the differentiation of 3T3-L1 preadipocytes to adipocytes, Mol Cell Proteomics 1, 213-222
28. Mauri, P. L. (2003), Proteomics studies based on microchromatography coupled to tandem mass spectrometry and new algorithm (MAProMa) for data handling of 2DC-MS/MS results, In 16th International Mass Spectrometry Conference, Edinburgh.
29. Akaogi, K., Okabe, Y., Sato, J., Nagashima, Y., Yasumitsu, H., Sugahara, K., and Miyazaki, K. (1996), Specific accumulation of tumor-derived adhesion factor in tumor blood vessels and in capillary tube-like structures of cultured vascular endothelial cells, Proc Natl Acad Sci USA 93, 8384-8389
30. Cattaneo, M., Orlandini, S., Beghelli, S., Moore, P. S., Sorio, C., Bonora, A., Bassi, C., Talamini, G., Zamboni, G., Orlandi, R., et al (2003), SELL L expression in pancreatic adenocarcinoma parallels SMAD4 expression and delays tumor growth in vitro and in vivo, Oncogene 22, 6359-6368 Albo, D., Berger, D. H., and Tuszynski, G. P. (1998) The effect of thrombospondin-1 and TGF-beta 1 on pancreatic cancer cell invasion. J Surg Res 76, 86-90
31. Cantero, D., Friess, H., Deflorin, J., Zimmermann, A., Brundler, M. A., Riesle, E., Korc, M., and Buchler, M. W. (1997) Enhanced expression of urokinase plasminogen activator and its receptor in pancreatic carcinoma. Br J Cancer 75, 388-395
32. Cattaruzza, S., Schiappacassi, M., Ljungberg-Rose, A., Spessotto, P., Perissinotto, D., Morgelin, M., Mucignat, M. T., Colombatti, A., and Perris, R. (2002) Distribution of PG-M/versican variants in human tissues and de novo expression of isoform V3 upon endothelial cell activation, migration, and neoangiogenesis in vitro. J Biol Chem 277, 47626-47635
33. Wight, T. N., Lara, S., Riessen, R., Le Baron, R., and Isner, J. (1997) Selective deposits of versican in the extracellular matrix of restenotic lesions from human peripheral arteries. Am J Pathol 151, 963-973
34. Aspberg, A., Adam, S., Kostka, G., Timpl, R., and Heinegard, D. (1999) Fibulin-1 is a ligand for the C-type lectin domains of aggrecan and versican. J Biol Chem 274, 20444-20449
35. Isogai, Z., Aspberg, A., Keene, D. R., Ono, R. N., Reinhardt, D. P., and Sakai, L. Y. (2002) Versican interacts with fibrillin-1 and links extracellular microfibrils to other connective tissue networks. J Biol Chem 277, 4565-4572
36. Crnogorac-Jurcevic, T., Efthimiou, E., Capelli, P., Blayeri, E., Baron, A., Terris, B., Jones, M., Tyson, K., Bassi, C., Scarpa, A., et al (2001) Gene expression profiles of pancreatic cancer and stromal desmoplasia. Oncogene 20, 7437-7446
37. Pejler, G., Winberg, J. O., Vuong, T. T., Henningsson, F., Uhlin-Hansen, L., Kimata, K., and Kolset, S. O. (2003) Secretion of macrophage urokinase plasminogen activator is dependent on proteoglycans. Eur J Biochem 270, 3971-3980
38. Sato, J., Hasegawa, S., Akaogi, K., Yasumitsu, H., Yamada, S., Sugahara, K., and Miyazaki, K. (1999) Identification of cell-binding site of angiomodulin (AGM/TAF/Mac25) that interacts with heparan sulfates on cell surface. J Cell Biochem 75, 187-195
39. Kishibe, J., Yamada, S., Okada, Y., Sato, J., Ito, A., Miyazaki, K., and Sugahara, K. (2000) Structural requirements of heparan sulfate for the binding to the tumor-derived adhesion factor/angiomodulin that induces cord-like structures to ECV-304 human carcinoma cells. J Biol Chem 275, 15321-15329
40. Nagakubo, D., Murai, T., Tanaka, T., Usui, T., Matsumoto, M., Sekiguchi, K., and Miyasaka, M. (2003) A high endothelial venule secretory protein, mac25/angiomodulin, interacts with multiple high endothelial venule-associated molecules including chemokines. J Immunol 171, 553-561
41. Adachi, Y., Itoh, F., Yamamoto, H., Arimura, Y., Kikkawa-Okabe, Y., Miyazaki, K., Carbone, D. P., and Imai, K. (2001) Expression of angiomodulin (tumor-derived adhesion factor/mac25) in invading tumor cells correlates with poor prognosis in human colorectal cancer. Int J Cancer 95, 216-222
42. Hill, A. S., MacCallum, P. K., Young, B. D., and Lillington, D. M. (2003) Molecular cloning of a constitutional t(7; 22) translocation associated with risk of hematological malignancy. Genes Chromosomes Cancer 38, 260-264
43. Sharma, B., Handler, M., Eichstefter, I., Whitelock, J. M., Nugent, M. A., and Iozzo, R. V. (1998) Antisense targeting of perlecan blocks tumor growth and angiogenesis in vivo. J Clin Invest 102, 1599-1608
44. Liu, W., Litwack, E. D., Stanley, M. J., Langford, J. K., Lander, A. D., and Sanderson, R. D. (1998) Heparan sulfate proteoglycans as adhesive and antiinvasive molecules. Syndecans and glypican have distinct functions. J Biol Chem 273, 22825-22832
45. 48. Zhu, Y., Xu, G., Patel, A., McLaughlin, M. M., Silverman, C., Knecht, K., Sweitzer, S., Li, X., McDonnell, P., Mirabile, R., et al (2002) Cloning, expression, and initial characterization of a novel cytokine-like gene family, Genomics 80, 144-150
46. Hansel, D. E., Rahman, A., Wehner, S., Herzog, V., Yeo, C. J., and Maitra, A. (2003) Increased expression and processing of the Alzheimer amyloid precursor protein in pancreatic cancer may influence cellular proliferation. Cancer Res 63, 7032-7037
47. Tamura, M., Nishizaka, S., Maeda, Y., Ito, M., Harashima, N., Harada, M., Shichijo, S., and Itoh, K. (2001) Identification of cyclophilin B-derived peptides capable of inducing histocompatibility leukocyte antigen-A2-restricted and tumor-specific cytotoxic T lymphocytes. Jpn J Cancer Res 92, 762-767
48. Gordon, J., Wu, C. H., Rastegar, M., and Safa, A. R. (2003) Beta2-microglobulin induces caspase-dependent apoptosis in the CCRF-HSB-2 human leukemia cell line independently of the caspase-3, -8 and -9 pathways but through increased reactive oxygen species. Int J Cancer 103, 316-327
49. Pilon, M., Peng, X. R., Spence, A. M., Plasterk, R. H., and Dosch, H. M. (2000) The diabetes autoantigen ICA69 and its Caenorhabditis elegans homologue, ric-19, are conserved regulators of neuroendocrine secretion. Mol Biol Cell 11, 3277-3288

1. A method for diagnosis and/or prognosis of neoplasias in animals, particularly of the type associated to a protein release in tumor cell microenvironment, wherein the method comprises at least the steps of:
drawing at least one sample from the patient;
determining the amount of a biomarker in said at least one sample drawn from the patient,
wherein said biomarker is a protein released by pancreatic cells.

2. Method as claimed in claim 1, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

3. Method as claimed in claim 1, wherein the neoplasia is a tumor of the pancreas.

4. Method as claimed in claim 1, wherein the neoplasia is selected from the group consisting of tumors of the breast, esophagus, head and neck, liver, lung, gastrointestinal tract, prostate, skin, kidney and/of urogenital system, metastases, micrometastases or a combination thereof.

5. Method as claimed in claim 1, wherein the animal is a mammal.

6. Method as claimed in claim 1, wherein said sample is a body fluid.

7. Method as claimed in claim 6, wherein said body fluid is selected from the group consisting of blood, plasma, serum, urine, sperm, interstitial fluid, spinal fluid or a combination thereof.

8. Method as claimed in claim 1, wherein the concentration of said biomarker is compared with known concentrations of the same biomarker, detected on samples of the same nature from different animals not suffering from neoplasia.

9. Method as claimed in claim 8, wherein said patients not suffering from neoplasia are animals having a benign tumor.

10. Method as claimed in claim 1, wherein the prognosis and/or diagnosis of the neoplasia is determined by comparing the concentration of said biomarker detected on samples drawn from the same patient.

11. A kit for diagnosis and/or prognosis of neoplasias in animals, particularly for carrying out the method as claimed in claim 1, comprising a detectable agent linked to a biomarker, wherein said biomarker is a protein released by pancreatic cells.

12. Kit as claimed in the preceding claim 11, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

13. Kit as claimed in claim 11, wherein said detectable agent is selected from the group consisting of an anti-biomarker antibody, a receptor for said biomarker, or functional fragments, or a combination thereof.

14. Kit as claimed in claim 11, wherein said anti-biomarker antibody is of the monoclonal or polyclonal type.

15. Kit as claimed in claim 11, wherein said agent is detectable by measuring chromatography, electrical capacitance, fluorescence, luminescence, mass, molecular weight, radioactivity or a combination thereof.

16. A reagent for diagnosis and/or prognosis of neoplasias in animals, particularly for carrying out the method as claimed in claim 1, comprising a detectable agent linked to a biomarker, wherein said biomarker is a protein released by pancreatic cells.

17. Reagent as claimed in claim 16, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

18. Use of the proteins released by pancreatic cells as biomarkers for diagnosis and/or prognosis of neoplasias, particularly for carrying out the method as claimed in claim 1, from a sample drawn from an animal.

19. Use as claimed in claim 18, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

20. A reagent for diagnosis and/or prognosis of neoplasias in animals, particularly for forming the kit as claimed in claim 11, comprising a detectable agent linked to a biomarker, wherein said biomarker is a protein released by pancreatic cells.

21. Reagent as claimed in claim 20, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.

22. Use of the proteins released by pancreatic cells as biomarkers for diagnosis and/or prognosis of neoplasias, particularly for forming the kit as claimed in claim 11, from a sample drawn from an animal.

23. Use as claimed in claim 22, wherein said biomarker is selected from the group consisting of: CSPG2/versican, Mac25/angiomodulin, IGFBP-1, HSPG2/perlecan, syndecan 4, FAM3C, APLP2, cyclofilin B, beta2 microglobulin, ICA69.
